(19) United States
(12) Patent Application Publication
(10) Pub. No.: US 2023/0008217 A1
(43) Pub. Date: Jan. 12, 2023

(54) LIGATION-BASED GENE EDITING USING CRISPR NICKASE

(71) Applicant: Agilent Technologies, Inc., Santa Clara, CA (US)

(72) Inventors: Nicholas M. SAMPAS, Santa Clara (US); David TAUSSIG, Santa Clara (US)

(21) Appl. No.: 17/786,382

(22) PCT Filed: Dec. 17, 2020
(86) PCT No.: PCT/US2020/065696

§ 371 Date: Jun. 16, 2022

Related U.S. Application Data
(60) Provisional application No. 62/949,433, filed on Dec. 17, 2019.

(51) Int. Cl. C12N 9/00; C12N 9/22; C12N 15/113; C12N 15/10

(52) U.S. Cl. CPC C12N 9/93; C12N 9/22; C12N 15/102; C12N 15/113; C12N 2310/20

(57) ABSTRACT

Disclosed are compositions and methods for gene editing. The present disclosure relates to compositions and methods for gene editing using a Cas nickase to cleave a double-stranded nucleic acid sequence near a target site and a ligase to incorporate a nucleic acid into a double-stranded nucleic acid sequence. The present disclosure also provides reagents for use in the gene editing methods. The present disclosure further provides kits containing reagents for use in the gene editing methods.

### CROSS-REFERENCE TO RELATED APPLICATIONS

This application claims priority to U.S. Provisional Application Serial No. 62/949,433, filed on Dec. 17, 2019, the disclosure of which is hereby incorporated by reference in its entirety.

### FIELD OF THE DISCLOSURE

The present disclosure relates generally to compositions and methods for gene editing. More particularly, the present disclosure relates to compositions and methods for gene editing using a CRISPR nickase to cleave genomic DNA near a target site and a ligase to incorporate a nucleic acid into a double-stranded nucleic acid sequence. The present disclosure also relates to reagents useful in the methods. The present disclosure further relates to kits including reagents for gene editing using the methods of the apresent disclosure.

### BACKGROUND OF THE DISCLOSURE

CRISPR (clustered regularly interspaced short palindromic repeats) represents a family of DNA sequences found within the genomes of prokaryotic organisms. Since the first demonstrations of CRISPR/Cas mammalian genome editing in 2013, numerous researchers have harnessed its capability to modify genomes in living cells with precision, efficiency, and ease of use, far outstripping previous technologies. However, several potential hazards of CRISPR-based genome editing have been observed, which compromise the safety and efficacy of this technology for clinical applications.

Three major classes of hazards associated with CRISPR addressed by the present disclosure are off-target editing, chromosomal rearrangements, and unintended indel (i.e., insertion or deletion) formation. Off-target editing occurs when the targeting portion of the CRISPR system, being the sequence of the typically 20-nucleotide “guide sequence” of the guide RNA (gRNA), anneals with sufficient stability to an unintended genomic target to allow the Cas protein to carry out its nuclease activity and cut the genome at unintended loci. Many CRISPR systems, such as the wild type S. pyogenes CRISPR/Cas9 system, produce a double stranded break (DSB) in the genome. This cleavage event is often repaired by non-homologous end-joining or microhomology mediated repair, both of which often result in indel formation, which is a desirable outcome for gene knockout applications. However, this double-strand break can result in undesired genomic rearrangements such as translocations, particularly with nonspecific or multiplexed editing. Unintended indel formation is also an unwanted but frequent side-effect of HDR applications designed to make precise edits to the target genome.

There have been several strategies to mitigate these hazards, with varying degrees of success. One class of CRISPR improvements have focused on improving the targeting specificity by altering either the Cas protein or gRNA components. These help to prevent off-target editing, but fail to improve unintended effects caused by DSBs. Three main strategies have been popularized to carry out CRISPR/Cas-based editing without DSBs: base-editing, nickase Cas mutants, and recently Prime editing.

Base editing helps to avoid indels and chromosomal rearrangements by using a modified Cas protein (typically a null variant) fused to another enzyme, such as cytosine deaminase, to directly change a base without cleaving the ribose backbone of DNA. A second strategy for avoiding DSBs involves other Cas variants termed nickases, which cut only one strand of the genome. This enables editing through HDR, but avoids the deleterious effects of DSBs.

A recent strategy, called Prime editing, was developed to improve the editing capability of CRISPR/Cas while avoiding DSBs. This technique involves fusion of a Cas nickase variant to a reverse transcriptase, which copies the intended edit from an elongated gRNA, and the intended edit is incorporated into the genome via the activity of endogenous FEN1 and DNA ligase. Prime editing, however, does not permit incorporation of modified or non-canonical bases.It would be advantageous to provide alternative methods of gene editing.

### BRIEF DESCRIPTION OF THE DISCLOSURE

Generally, the present disclosure provides methods and variations involving using a CRISPR nickase to cleave genomic DNA near a target site. The methods utilize four main components: a CRISPR nickase (also known as Cas nickase) and three polynucleotides, including a CRISPR guide RNA sequence, a splint oligonucleotide and a ligand oligonucleotide.

In one aspect, the present disclosure is directed to a method of editing a double-stranded nucleic acid comprising a target strand and a displaced strand that is complementary to the target strand. The method includes: providing to the double-stranded nucleic acid: a Cas nickase; a CRISPR guide RNA that includes a guide sequence that hybridizes with the target strand of the double-stranded nucleic acid; a ligand oligonucleotide that includes a nucleic acid sequence to be incorporated into the double-stranded nucleic acid; a splint oligonucleotide that includes a first nucleic acid sequence that hybridizes with the ligand oligonucleotide and a second nucleic acid sequence that hybridizes with the displaced strand of the double-stranded nucleic acid; wherein the CRISPR guide RNA and Cas nickase form a complex with the double-stranded nucleic acid and make a single-strand cut in the displaced strand to form a nick in the displaced strand, and the splint oligonucleotide hybridizes to the displaced strand adjacent to the nick, and the ligand oligonucleotide hybridizes to the splint oligonucleotide; and ligating one end of the ligand oligonucleotide to a nucleotide adjacent to the nick in the displaced strand to thereby result in the ligand oligonucleotide to be incorporated into the double-stranded nucleic acid.

The CRISPR guide RNA sequence, the splint oligonucleotide and/or the ligand oligonucleotide may reside in the same nucleic acid molecule. In one aspect, the present disclosure is directed to a CRISPR guide RNA including: a guide sequence that hybridizes with a nucleic acid sequence of a double-stranded nucleic acid sequence; a scaffold nucleic acid sequence; and at least one of a ligand oligonucleotide and a splint oligonucleotide.

In one aspect, the present disclosure is directed to a CRISPR guide RNA including: a guide sequence that hybridizes with a double-stranded nucleic acid sequence; a scaffold nucleic acid sequence; at least one of an inverted base linker and a cleavable linker; and at least one of a ligand oligonucleotide and a splint oligonucleotide.

In one aspect, the present disclosure is directed to a kit for ligation-based gene editing. The kit includes: a CRISPR guide RNA that includes: a guide sequence that hybridizes with a double-stranded nucleic acid sequence; and at least one of a ligand oligonucleotide and a splint oligonucleotide; and instructions for performing ligation-based gene editing.

In one aspect, the present disclosure is directed to a CRISPR guide RNA including: a guide sequence that hybridizes with a nucleic acid sequence of a double-stranded nucleic acid sequence; and at least one of a ligand oligonucleotide and a splint oligonucleotide.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1A depicts a complex comprising a Cas nickase (shaded in purple) and CRISPR guide RNA (“Guide RNA” depicted in light blue) with a 20-base guide sequence hybridized to a targeted strand of genomic DNA (gDNA; depicted in black) above the displaced strand of gDNA, which includes a protospacer adjacent motif (PAM, depicted in red).

FIG. 1B depicts the position of the cut (“nick” green arrow) introduced in the gDNA by the Nickase (not shown) complexed with the CRISPR guide RNA (depicted in light blue) near a PAM sequence (depicted in red) in the gDNA.

FIG. 2 depicts a CRISPR guide RNA (light blue) hybridized with a target strand of gDNA in the gDNA and a splint oligonucleotide (“Splint”; light blue) with regions hybridizing to the displaced strand of gDNA and a ligand oligonucleotide (“Ligand”; purple) and illustrating the nick located between the 3′ end of the displaced strand of the gDNA and the 5′ end of the ligand oligonucleotide.

FIG. 3 depicts a CRISPR guide RNA (light blue) hybridized with a target strand of gDNA and a splint oligonucleotide (hybridized to a displaced strand of the gDNA) coupled with a ligand oligonucleotide in a hairpin-shaped structure and illustrating the nick located between the 3′ end of the gDNA of the displaced strand and the 5′ end of the ligand oligonucleotide.

FIG. 4 depicts a CRISPR guide RNA (light blue) hybridized with a target strand of gDNA and a splint oligonucleotide coupled with a ligand oligonucleotide in a hairpin-shaped DNA structure and including a photocleavable linker. The splint oligonucleotide is hybridized with a displaced strand of gDNA. The nick is located between the 3′ end of the gDNA of the displaced strand and the 5′ end of the ligand oligonucleotide.

FIG. 5 depicts ligation of the 3′ end of the gDNA with the 5′ end of the hairpin structured ligand oligonucleotide-splint oligonucleotide (including a photocleavable linker) by a DNA ligase.

FIG. 6 depicts illumination of the sample with 365 nm wavelength UV light.

FIG. 7 depicts the structure following cleavage of the hairpin of the hairpin structured ligand oligonucleotide-splint oligonucleotide of FIGS. 3-6.

FIG. 8 depicts a 3′ flap and a 5′ flap remaining following cleavage.

FIG. 9 depicts cleavage of the 5′ flap by endogenous FEN1, for example, leaving a nick that is ultimately repaired by endogenous DNA-ligase, also leaving and unresolved heteroduplex.

FIG. 10 depicts repair of the nick by an endogenous ligase. Additionally, the heteroduplex (illustrated in FIG. 9) is resolved by DNA repair mechanisms.

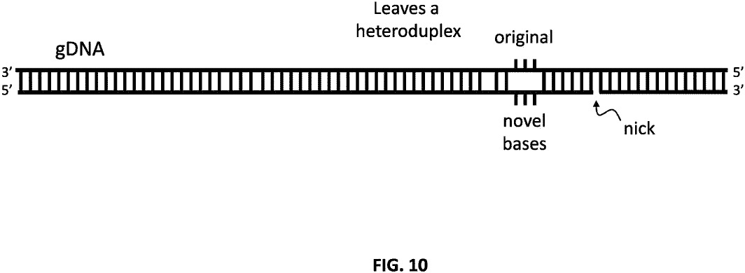

FIG. 11 depicts an embodiment of a method using two CRISPR guide RNA molecules to introduce two nicks in a gDNA target and insertion of a ligand oligonucleotide by displacement of one strand of the gDNA. As shown, the two CRISPR guide RNA molecules target different sequences in a gDNA whereby the Cas nickase makes two separate cuts in the gDNA resulting in two nicks in the same strand of the gDNA. A splint oligonucleotide hybridizes with the displaced strand at a first target site and a ligand oligonucleotide having a sequence length matching the number of bases between the two nicks hybridizes to the splint oligonucleotide. The nicked gDNA of the displaced strand is displaced by the ligand oligonucleotide and two ligation events results in the incorporation of the ligand oligonucleotide into the gDNA. Edits included in the ligand oligonucleotide are incorporated into the gDNA locus.

FIG. 12 depicts an embodiment of a CRISPR guide RNA coupled via an inverted base linker to a hairpin structured splint oligonucleotide-ligand oligonucleotide, and also including a photocleavable linker.

FIG. 13 depicts an embodiment of a CRISPR guide RNA coupled via an inverted base linker to a ligand oligonucleotide, and also including a photocleavable linker.

FIG. 14 depicts an embodiment illustrating a CRISPR guide RNA hybridized with a first strand of a double stranded gDNA, a splint oligonucleotide (RNA or DNA) hybridized with a second strand of the double-stranded gDNA and hybridized with a ligand oligonucleotide following cleavage of the second nucleic acid strand of the double-stranded gDNA.

FIG. 15 depicts an embodiment of a CRISPR guide RNA coupled to a splint oligonucleotide, wherein the CRISPR guide RNA is hybridized with a first strand of a double stranded gDNA, the splint oligonucleotide (RNA) is hybridized with a second strand of the double-stranded gDNA and hybridized with a ligand oligonucleotide (DNA) following cleavage of the second nucleic acid strand of the double-stranded gDNA and showing the nick between the 3′ end of the gDNA and the 5′ end of the ligand oligonucleotide.

FIG. 16 depicts an embodiment of a CRISPR guide RNA coupled to a splint oligonucleotide (RNA) and the splint oligonucleotide is coupled to a ligand oligonucleotide (DNA) via an inverted base linker and includes a photocleavable linker at the 3′ end of the ligand oligonucleotide, wherein the CRISPR guide RNA is hybridized with a first strand of a double stranded gDNA, the splint oligonucleotide (RNA) is hybridized with a second strand of the double-stranded gDNA and hybridized with the ligand oligonucleotide (DNA) following cleavage of the second nucleic acid strand of the double-stranded gDNA and showing the nick between the 3′ end of the gDNA and the 5′ end of the ligand oligonucleotide.

FIG. 17 depicts an embodiment of a CRISPR guide RNA coupled to a splint oligonucleotide (RNA) and ligand oligonucleotide. The CRISPR guide RNA is coupled to the splint oligonucleotide (RNA) via an inverted-base linker. The splint oligonucleotide is coupled to the ligand oligonucleotide (DNA) via a photocleavable linker and includes a hairpin structure. The CRISPR guide RNA is hybridized with a first strand of a double stranded gDNA, the splint oligonucleotide (RNA) is hybridized with a second strand of the double-stranded gDNA and hybridized with the ligand oligonucleotide (DNA) following cleavage of the second nucleic acid strand of the double-stranded gDNA and showing the nick between the 3′ end of the gDNA and the 5′ end of the ligand oligonucleotide.

FIGS. 18A and 18B depict the results of Example 1. FIG. 18A is a schematic illustrating both strands of the target DNA (i) with the bottom strand containing a site Δ for cleavage by Cas9 H840A Nickase. The cleavage and ligation reactions produced 3 novel strands: ii) the larger fragment of the cleaved target DNA ligated to the ligand (in red), iii) the larger fragment of the target DNA cleaved but not ligated, and iv) the smaller fragment of the cleaved target DNA. FIG. 18B depicts an image of a Urea-PAGE gel showing bands corresponding to the fragments described in FIG. 18A in 3 separate treatments: 1) target DNA + Non-targeting gRNA + Cas9H840A; 2) target DNA + targeting gRNA + Cas9H840A + splint + ligand; 3) target DNA + targeting gRNA + Cas9H840A + splint + ligand + T3 Ligase.

### DETAILED DESCRIPTION

Unless defined otherwise, all technical and scientific terms used herein have the same meaning as commonly understood by one of ordinary skill in the art to which the disclosure belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid synthesis. The techniques and procedures are generally performed according to conventional methods in the art and various general references. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.”

As used herein, a “nucleic acid” sequence means a DNA or RNA sequence, or a mix of DNA and RNA. The term encompasses sequences that include natural nucleotides and any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine. Nucleic acids may be of genomic or synthetic origin and may be single- or double-stranded. Polynucleotides and oligonucleotides are within the scope of nucleic acids.

As used herein, “recombinant,” when used in connection with a nucleic acid molecule, refers to a molecule that has been created or modified through deliberate human intervention by genetic engineering. For example, a recombinant nucleic acid molecule is one having a nucleotide sequence that has been modified to include an artificial nucleotide sequence or to include some other nucleotide sequence that is not present within its native (non-recombinant) form. Further, a recombinant nucleic acid molecule has a structure that is not identical to that of any naturally occurring nucleic acid molecule or to that of any fragment of a naturally occurring genomic nucleic acid molecule spanning more than one gene. A recombinant nucleic acid molecule also includes, without limitation, (a) a nucleic acid molecule having a sequence of a naturally occurring genomic or extrachromosomal nucleic acid molecule, but which is not flanked by the coding sequences that flank the sequence in its natural position; (b) a nucleic acid molecule incorporated into a construct, expression cassette or vector, or into a host cell’s genome such that the resulting polynucleotide is not identical to any naturally occurring vector or genomic DNA; (c) a separate nucleic acid molecule such as a cDNA, a genomic fragment, a fragment produced by polymerase chain reaction (PCR) or a restriction fragment; and (d) a recombinant nucleic acid molecule having a nucleotide sequence that is part of a hybrid gene (i.e., a gene encoding a fusion protein). As such, a recombinant nucleic acid molecule can be modified (chemically or enzymatically) or unmodified DNA or RNA, whether fully or partially single-stranded or double-stranded or even triple-stranded.

Methods for synthesizing nucleic acid molecules are well known in the art, such as cloning and digestion of the appropriate sequences in genetic engineering, as well as direct chemical synthesis (e.g., ink-jet deposition and electrochemical synthesis). Methods of cloning nucleic acid molecules are described, for example, in Ausubel et al. (1995), supra; Copeland et al. (2001) Nat. Rev. Genet. 2:769-779; PCR Cloning Protocols, 2nd ed. (Chen & Janes eds., Humana Press 2002); and Sambrook & Russell (2001), supra. Methods of direct chemical synthesis of nucleic acid molecules include, but are not limited to, the phosphotriester methods of Reese (1978) Tetrahedron 34:3143-3179 and Narang et al. (1979) Methods Enzymol. 68:90-98; the phosphodiester method of Brown et al. (1979) Methods Enzymol. 68:109-151; the diethylphosphoramidate method of Beaucage et al. (1981) Tetrahedron Lett. 22:1859-1862; and the solid support methods of Fodor et al. (1991) Science 251:767-773; Pease et al. (1994) Proc. Natl. Acad. Sci. USA 91:5022-5026; and Singh-Gasson et al. (1999) Nature Biotechnol. 17:974-978; as well as U.S. Pat. No. 4,485,066. See also, Peattie (1979) Proc. Natl. Acad. Sci. USA 76:1760-1764; as well as EP Patent No. 1 721 908; Int’l Patent Application Publication Nos. WO 2004/022770 and WO 2005/082923; U.S. Pat. Application Publication No. 2009/0062521; and U.S. Pat. Nos. 6,521,427; 6,818,395 and 7,521,178.

For nucleotide (and nucleic acid) sequences, “variant” refers to a similar but not identical nucleotide sequence to a reference nucleotide sequence. For nucleotide sequences, a variant includes a nucleotide sequence having deletions (i.e., truncations) at the 5′ and/or 3′ end, deletions and/or additions of one or more nucleotides at one or more internal sites compared to the nucleotide sequence of the reference nucleic acid molecules as described herein; and/or substitution of one or more nucleotides at one or more sites compared to the nucleotide sequence of the reference nucleic acid molecules described herein. In some embodiments, variants are constructed in a manner to maintain the open reading frame.

Naturally occurring allelic variants can be identified by using well-known molecular biology techniques such as, for example, polymerase chain reaction (PCR) and hybridization techniques. Variant nucleotide sequences also can include synthetically derived sequences, such as those generated, for example, by site-directed mutagenesis but which still provide a functionally active modified protein. Generally, variants of a nucleotide sequence of the reference nucleic acid molecules as described herein will have at least about 70%, 75%, 80%, 85%, 90%, 91%, 92%, 93%, 94%, 95%, 96%, 97%, 98%, 99% or more sequence identity to the nucleotide sequence of the reference nucleic acid molecules as determined by sequence alignment programs and parameters as described elsewhere herein.

Methods of mutating and altering nucleotide sequences, as well as DNA shuffling, are well known in the art. See, Crameri et al. (1997) Nature Biotech. 15:436-438; Crameri et al. (1998) Nature 391:288-291; Kunkel (1985) Proc. Natl. Acad. Sci. USA 82:488-492; Kunkel et al. (1987) Methods in Enzymol. 154:367-382; Moore et al. (1997) J. Mol. Biol. 272:336-347; Stemmer (1994) Proc. Natl. Acad. Sci. USA 91:10747-10751; Stemmer (1994) Nature 370:389-391; Zhang et al. (1997) Proc. Natl. Acad. Sci. USA 94:4504-4509; and Techniques in Molecular Biology (Walker & Gaastra eds., MacMillan Publishing Co. 1983) and the references cited therein; as well as U.S. Pat. Nos. 4,873,192; 5,605,793 and 5,837,458. As such, the nucleic acid molecules as described herein can have many modifications.

Variants of the reference nucleic acid molecules described herein also can be evaluated by comparing the percent sequence identity between the polypeptide encoded by a variant and the polypeptide encoded by the reference nucleic acid molecule. Thus, for example, an isolated nucleic acid molecule can be one that encodes a polypeptide with a given percent sequence identity to the polypeptide of interest. Percent sequence identity between any two polypeptides can be calculated using sequence alignment programs and parameters described elsewhere herein. Where any given pair of polynucleotides of the present disclosure is evaluated by comparison of the percent sequence identity shared by the two polypeptides they encode, the percent sequence identity between the two encoded polypeptides can be at least about 70%, 75%, 80%, 85%, 90%, 91%, 92%, 93%, 94%, 95%, 96%, 97%, 98%, 99% or more sequence identity.

Determining percent sequence identity between any two sequences can be accomplished using a mathematical algorithm. Non-limiting examples of such mathematical algorithms include, but are not limited to, the algorithm of Myers & Miller (1988) CABIOS 4:11-17; the local alignment algorithm of Smith et al. (1981) Adv. Appl. Math. 2:482-489; the global alignment algorithm of Needleman & Wunsch (1970) J. Mol. Biol. 48:443-453; the search-for-local alignment method of Pearson & Lipman (1988) Proc. Natl. Acad. Sci. USA 85:2444-2448; the algorithm of Karlin & Altschul (1990) Proc. Natl. Acad. Sci. USA 87:2264-2268, modified as in Karlin & Altschul (1993) Proc. Natl. Acad. Sci. USA 90:5873-5877.

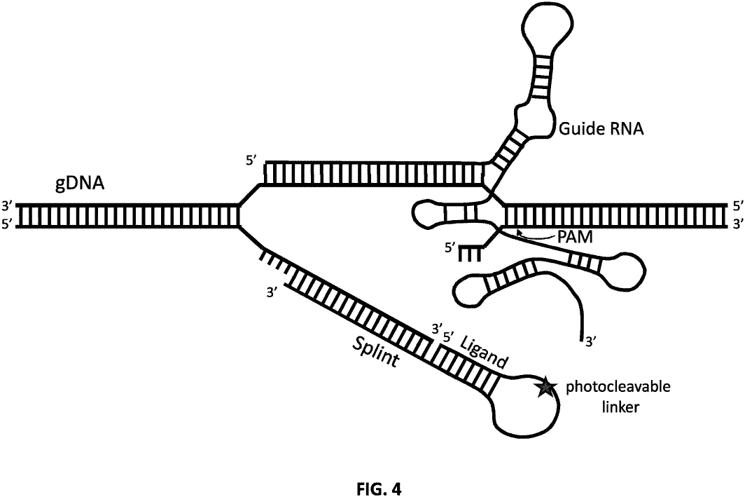

As used herein, “coupled” refers to being joined as part of the same molecule. For example, and as described in more detail below, where a splint oligonucleotide is coupled to a CRISPR guide RNA by an inverted-base linker, the inverted-base linker joins the splint oligonucleotide to the CRISPR guide RNA such that the splint oligonucleotide, inverted-base linker and the CRISPR guide RNA are in the form of a single molecule. Similarly, for example, where a splint oligonucleotide is coupled to a ligand oligonucleotide, the splint oligonucleotide and the ligand oligonucleotide are in the form of a single molecule. FIG. 4 illustrates an exemplary embodiment of a splint oligonucleotide coupled with a ligand oligonucleotide in a hairpin-shaped DNA structure and including a photocleavable linker all as part of a single molecule.

The compositions and methods of the present disclosure improves upon the flexibility of gene editing relative to most existing methods. Without being bound by theory, in general, the compositions and methods of the present disclosure introduce a nucleic acid sequence (a “ligand oligonucleotide”) by taking advantage of a cell’s natural DNA repair mechanisms. Briefly, following a single-stranded cut to a double-stranded nucleic acid (e.g., a genomic DNA), a cell resolves the cut by ligating the 3′ end of the double stranded nucleic acid to the 5′ end of the ligand oligonucleotide at the site of the single-stranded cut, leaving (in some cases) a 5′ flap and a 3′ flap. After resolving the 5′ flap and 3′ flap, the DNA repair mechanisms resolve the heteroduplex to incorporate the ligand oligonucleotide into the double-stranded nucleic acid.

As used herein, “hybridization” and “hybridize(s)” refer to a process by which a strand of nucleic acid binds with a complementary (completely or partially complementary) strand through base pairing.

As used herein, “CRISPR guide RNA” or “gRNA” refers to any one of many natural or modified nucleic acid sequences comprising a 5′ region termed the “guide” sequence, which hybridizes to a target strand of double-stranded nucleic acid (such as, for example, a genomic DNA sequence (gDNA)) and a scaffold nucleic acid sequence. The guide sequence can range from about 17 nucleotides in length to about 30 nucleotides in length, including about 17 nucleotides to about 20 nucleotides, including about 18 nucleotides to about 25 nucleotides, and including about 21 nucleotides to about 24 nucleotides. CRISPR guide RNAs can be a single guide RNA (“sgRNA”), where the guide sequence and scaffold are in a single molecule. In dual guide RNAs, the guide sequence and scaffold may be in two molecules linked by hybridization (or the guide sequence and part of the scaffold in one molecule while the remaining scaffold in a second molecule). The CRISPR guide RNA scaffold region (also referred to herein as “a scaffold nucleic acid sequence”) is recognized by and provides a binding site for a CRISPR protein (e.g., a Cas protein, including a Cas nickase (e.g., a Cas9 nickase)). The scaffold nucleic acid sequence of the CRISPR guide RNA forms a complex with the Cas nickase to make a single-strand cut (i.e., a nick) . CRISPR guide RNA(s) can be produced in vitro (i.e., synthesized and purified) using a variety of RNA production techniques known to those skilled in the art, such as in vitro transcription or chemical synthesis. CRISPR guide RNA(s) can also be expressed from DNA, e.g., after transfection of cells with a plasmid that expresses the guide RNA(s).As used herein, “nick” refers to a single-stranded cut (or break) in a double-stranded nucleic acid sequence. Some Cas nickases nick the target strand, which is recognized and hybridized by the CRISPR guide RNA, while other Cas nickases nick the strand that opposes the target strand. The strand opposite from the target strand is known as the displaced strand and contains a protospacer adjacent motif (PAM) site. The Cas nickases useful in the present invention nick the displaced strand.

As used herein, “Cas nickase” refers to a CRISPR-associated (Cas) nuclease that mediates cleavage of only a single strand of a defined nucleotide sequence. Here, the Cas nickase cuts the displaced strand of the nucleic acid sequence to introduce a nick. Suitable Cas nickases include, for example, Cas9 nickase and Cpf1 nickase. Cas nickases can be found in nature or developed by modification of natural CRISPR nucleases. Cas nickases found in nature can be isolated and purified using a variety of protein isolation and purification techniques known to those skilled in the art. The Cas nickase can be produced recombinantly, or produced in vitro (i.e., synthesized and purified) using a variety of protein production techniques known to those skilled in the art. The Cas nickase can be provided as a protein or mRNA transcript. The Cas nickase can alternatively be provided as a nucleotide vector capable of expressing the Cas nickase. In one aspect, a ligase is fused to a Cas9 nickase as a fusion protein. Without being bound by theory, the fusion of the ligase and Cas nickase can substantially accelerate the ligation reaction over an endogenous ligase because of the close proximity afforded by targeting the fused ligase to the reaction site bound by the Cas9 protein or other CRISPR-related protein molecules. Similarly, the ligase can be fused to the guide RNA.

As used herein, “splint oligonucleotide” and “splint sequence” refer to a polynucleotide sequence having a first nucleic acid sequence that can hybridize with the displaced strand of double-stranded nucleic acid (e.g., genomic DNA) and a second nucleic acid sequence that can hybridize with the donor sequence, which is to be incorporated into the double-stranded nucleic acid by ligation (the donor sequence is part of the “ligand oligonucleotide”). The splint sequence can be RNA, DNA, and combinations thereof.

As used herein, “ligand oligonucleotide” and “ligand sequence” refer to a polynucleotide sequence comprising a donor sequence to be incorporated into the displaced strand of double-stranded nucleic acid by ligation. This polynucleotide may include natural bases or modified bases, such as methylated bases, inverted bases, photocleavable linkers, and phosphothioate linkers, and is typically between about 10 nucleotides and about 30 nucleotides in length, but may include as few as 1 (when concatenated to a guide or splint) and as many as about 100 nucleotides. Other modifications may be used to increase its stability in the cell, or provide one or more cleavage site(s). Methylated bases may be useful for applications where changing the methylation state of a genomic locus is useful. The region of the ligand oligonucleotide to be incorporated into the target site should be DNA.

In one aspect, the present disclosure is directed to a method of editing a double-stranded nucleic acid, the method comprising: providing to the double-stranded nucleic acid: a Cas nickase; a CRISPR guide RNA comprising a guide sequence that hybridizes with a target strand of the double-stranded nucleic acid and a scaffold nucleic acid sequence; a ligand oligonucleotide comprising a ligand nucleic acid sequence to be incorporated into the double-stranded nucleic acid; a splint oligonucleotide comprising a first nucleic acid sequence that hybridizes with the ligand oligonucleotide and a second nucleic acid sequence that hybridizes with a displaced strand of the double-stranded nucleic acid; wherein the CRISPR guide RNA and Cas nickase form a complex with the double-stranded nucleic acid and make a single-strand cut in the displaced strand to form a nick in the displaced strand, and the splint oligonucleotide hybridizes to the displaced strand adjacent to the nick, and the ligand oligonucleotide hybridizes to the splint oligonucleotide; and ligating one end of the ligand oligonucleotide to a nucleotide adjacent to the nick in displaced strand to thereby result in the ligand oligonucleotide to be incorporated into the double-stranded nucleic acid.

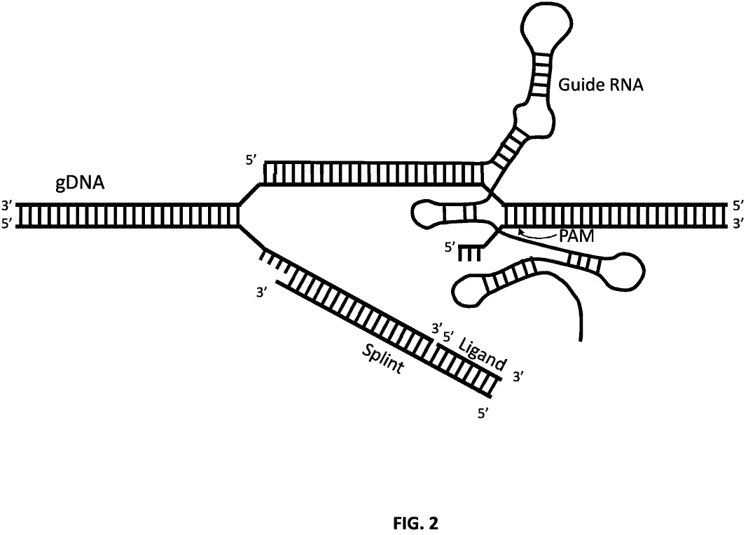

During the process, the splint oligonucleotide hybridizes to a long flap of the displaced strand adjacent to the nick (see e.g., FIG. 2; the long flap is the single-stranded region to the left of the nick). Due to the complementarity between the splint oligonucleotide and the ligand oligonucleotide, the ligand oligonucleotide is brought next to the long flap (see e.g., FIG. 2). The long flap and the ligand oligonucleotide are aligned along the splint oligonucleotide, with only a gap between the 3′ end of the long flap and the 5′ end of the ligand oligonucleotide. The gap is then ligated by a ligase. The long flap, now with the ligand oligonucleotide ligated to it, undergoes strand displacement and the ligand oligonucleotide is incorporated into the double-stranded nucleic acid, usually genomic DNA.

In one aspect, the CRISPR guide RNA, the ligand oligonucleotide, and the splint oligonucleotide are provided as separate molecules (as shown in FIG. 2).

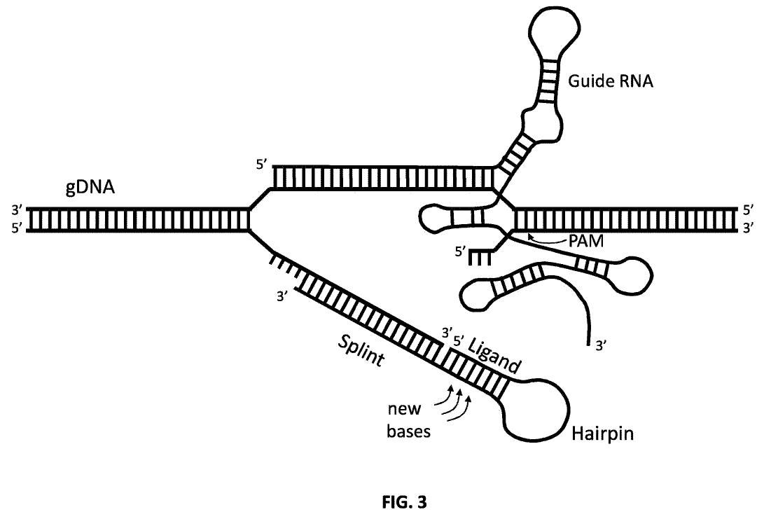

In some other embodiments, the ligand oligonucleotide and the splint oligonucleotide are coupled. The ligand oligonucleotide and the splint oligonucleotide can be coupled by covalent coupling between a terminal nucleic acid of the ligand oligonucleotide and a terminal nucleic acid of the splint oligonucleotide. As illustrated in FIG. 3, the ligand oligonucleotide and the splint oligonucleotide are coupled, and form a hairpin structure. The ligand oligonucleotide and the splint oligonucleotide can also be coupled using a linker. Suitable linkers include inverted base linkers, cleavable linkers, and combinations thereof. Exemplary linkers are described herein. As illustrated in FIG. 4, the ligand oligonucleotide and the splint oligonucleotide are coupled to form a hairpin structure and further include a cleavable linker. The ligand oligonucleotide and splint oligonucleotide can also be pre-hybridized through their complementary region.

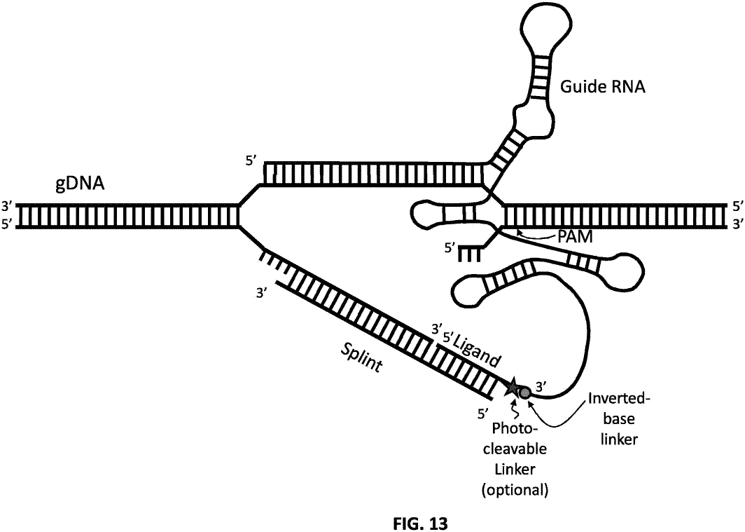

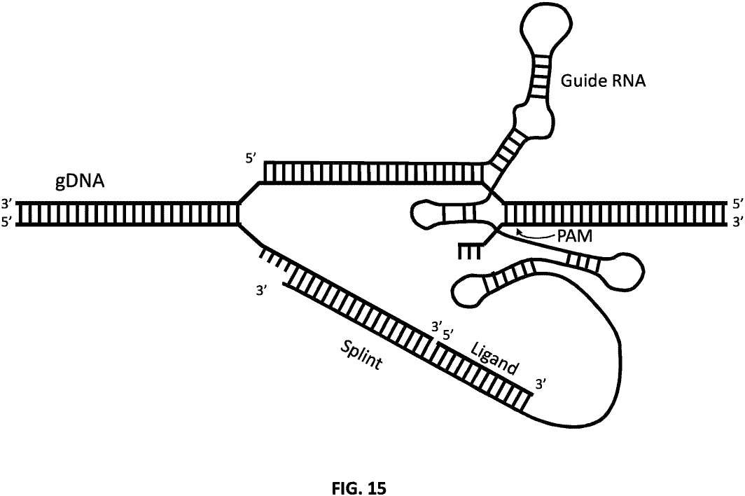

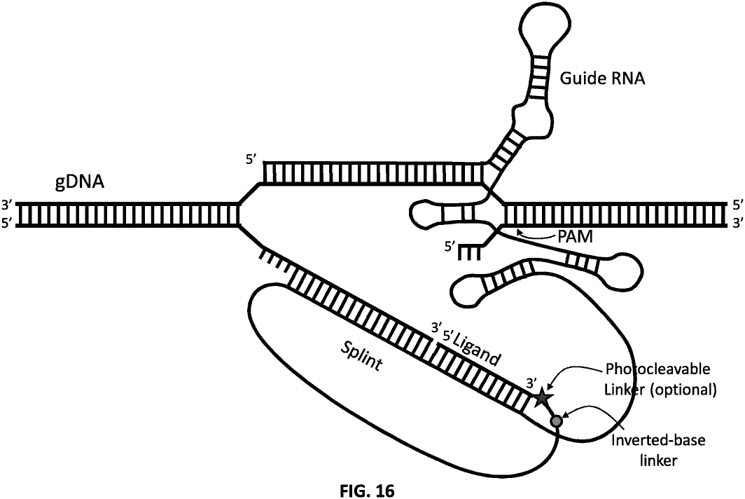

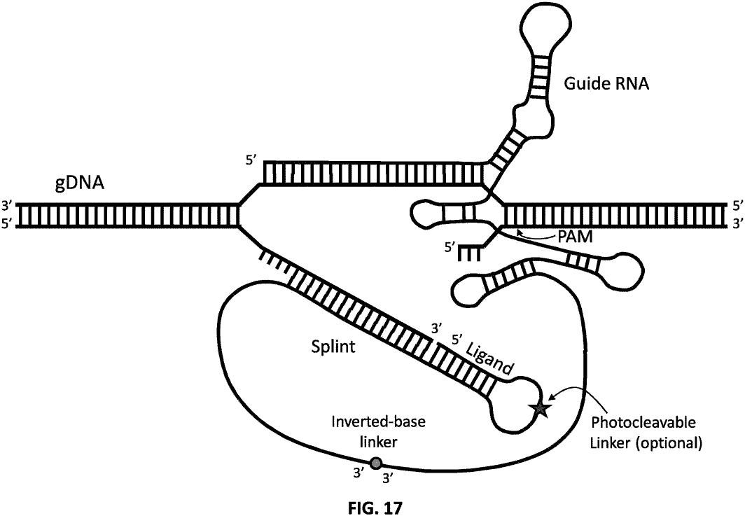

In one aspect, the ligand oligonucleotide is coupled to the CRISPR guide RNA, as illustrated in FIG. 13. In some other embodiments, the splint oligonucleotide is coupled to the CRISPR guide RNA. In one aspect, the ligand oligonucleotide and the splint oligonucleotide are coupled to the CRISPR guide RNA, as illustrated in FIG. 15. The ligand oligonucleotide, the splint oligonucleotide and the CRISPR guide RNA can be coupled through covalent attachment or hybridization. Examples of covalent coupling include, for example, phosphodiester bonding between a terminal nucleic acids of the ligand oligonucleotide, a terminal nucleic acid of the splint oligonucleotide, and a terminal nucleic acid of the CRISPR guide RNA. The ligand oligonucleotide, the splint oligonucleotide and the CRISPR guide RNA can be coupled using a linker, as illustrated in FIGS. 16 and 17. Suitable linkers include inverted base linkers, cleavable linkers, and combinations thereof. Exemplary linkers are described herein. FIG. 13 illustrates an embodiment where the CRISPR guide RNA and ligand oligonucleotide are coupled by both an inverted base linker and a cleavable linker. In this embodiment, the ligand oligonucleotide is coupled to the 3′ end of the CRISPR guide RNA and the splint oligonucleotide is coupled to the 3′ end of the CRISPR guide RNA. Suitable inverted base linkers are commercially available from Trilink (www.trilinkbiotech.com) and Integrated DNA Technologies (www.idtdna.com), for example. Inverted base linkers can be A, C, G, and T. Suitable inverted base linkers can be 3′-3′ reversed linkage and 5′-5′ reversed linkage as illustrated below. Suitable cleavable linkers include photocleavable linkers commercially available from Trilink (“PC-linker”) and Integrated DNA Technologies (e.g., “PC Spacer”) illustrated below.

The method can further include illuminating the sample with ultraviolet light. Exposure of the sample to ultraviolet light in the 300-370 nm spectral range of about 300 nm to about 370 nm can result in cleavage at the photocleavable linker.

The method can further include providing a flap endonuclease. In one aspect, the flap endonuclease is an endogenous flap endonuclease (i.e., a flap endonuclease that exists within a cell, for example). In another aspect, the flap endonuclease is an exogenous flap endonuclease. Suitable flap endonucleases include, for example, FEN1. An exogenous flap endonuclease can be prepared using synthetic methods and recombinant protein expression methods, for example. An exogenous flap endonuclease can be provided to a cell sample using the methods described herein. The flap endonuclease cut the 5′ and 3′ flaps flush with the double-stranded nucleic acid sequence.

The method can further include providing a ligase. In one aspect, the ligase is an endogenous ligase (i.e., a ligase that exists within a cell, for example). In another aspect, the ligase is an exogenous ligase. Suitable ligases include, for example, T4 ligase, T7 ligase, T3 ligase, and PBCV-1 DNA ligase from Chlorella virus (also known as Chlorella virus DNA Ligase), (SPLINTR®-Ligase commercially available from New England BioLabs, Inc.). An exogenous ligase can be prepared using synthetic methods and recombinant protein expression methods, for example. An exogenous ligase can be provided to a cell sample using any available methods. The ligase ligates the ligand oligonucleotide with the 3′ end of the displaced strand of the double-stranded nucleic acid sequence. An exogenous DNA ligase may be delivered into the cell in the form of a protein, messenger RNA, or plasmid DNA. Moreover, an exogenous DNA ligase may be delivered as a discrete unit or coupled to a Cas protein as a fusion protein. If delivered as a discrete unit, methods may be employed to recruit the DNA ligase to the target site. One example of such a method is the Casilio system (Cell Research (2016) 26:254-257), in which the DNA ligase is fused to a domain of the Pumilio protein and targeted to the target site via binding to an extension on the guide RNA. Another example (Nat Commun. 2017 Nov 23;8(1):1711) utilizes a streptavidin-binding aptamer coupled to the guide RNA to recruit a biotin-labeled protein, such as a DNA ligase, in the presence of streptavidin (which can be transfected into the cell). In some embodiments, the streptavidin is monomeric streptavidin. The streptavidin-binding aptamer may alternatively be coupled to the splint or the ligand oligonucleotide (but outside of the region to be incorporated into the target site).

In one aspect, the method of this invention is performed in vitro or ex vivo. In particular, the method may be performed in a cell or cells isolated from a subject, such as primary cells. In another aspect, the method is performed in vivo.

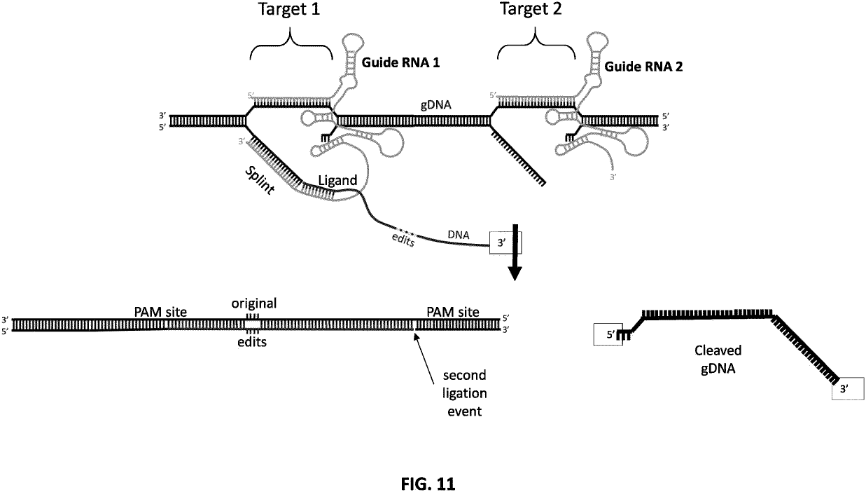

In another aspect, the method further includes providing a second CRISPR guide RNA that includes a guide sequence complementary to a second target sequence that is different from the target sequence to which the first CRISPR guide RNA hybridizes. As illustrated in FIG. 11, each CRISPR guide RNA complexes with a nickase to introduce two nicks in a displaced strand of the double-stranded nucleic acid. A splint oligonucleotide hybridizes to the displaced strand and to a ligand oligonucleotide. In this method, the ligand oligonucleotide is introduced into the displaced strand between the two nicks. The cleaved target nucleic acid sequence is displaced by the ligand oligonucleotide which is incorporated into the target nucleic acid sequence.

In the method, when all of the components are provided, the CRISPR guide RNA and Cas nickase form a complex with the double-stranded nucleic acid and make a single-strand cut in the displaced strand, which results in two DNA flaps opposite the complex formed by the guide RNA hybridized with the target sequence of the double stranded nucleic acid. The splint oligonucleotide hybridizes to the flap terminating in the 5′ base adjacent to the nick in the displaced strand, and the ligand oligonucleotide hybridizes to the splint oligonucleotide in such a way to bring the 3′ end of the splint in close proximity to the 5′ nucleotide of the flap. The 3′ nucleotide of the splint oligonucleotide is then ligated to the 5′ nucleotide of the flap to thereby result in the ligand oligonucleotide to be incorporated (by covalent linkage) into the double-stranded nucleic acid.

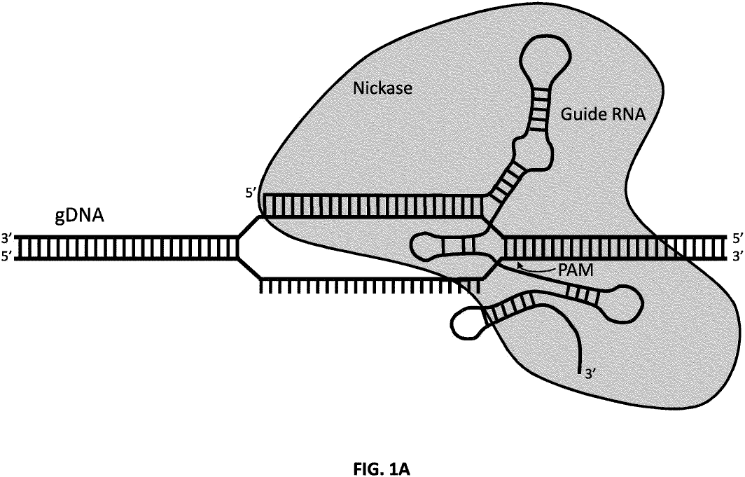

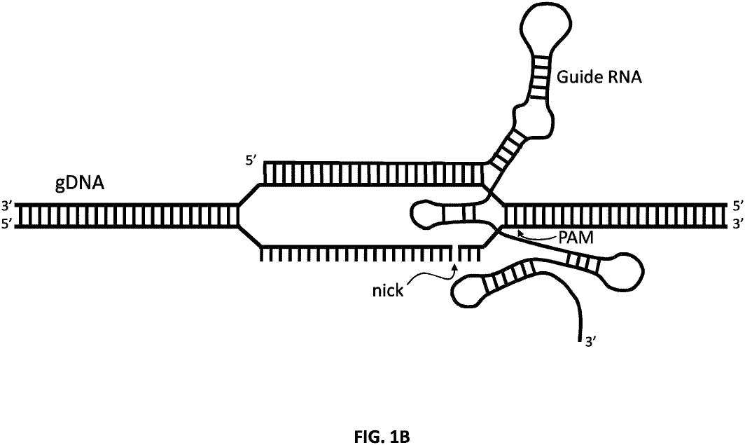

The methods of the present disclosure may be described by referring to the Figures. FIG. 1A illustrates the complex formed by a double-stranded genomic DNA, a CRISPR guide RNA and a nickase. The CRISPR guide RNA hybridizes via its guide sequence to a DNA recognition sequence (also referred to herein as the “target strand”) in the double-stranded nucleic acid (genomic DNA “gDNA” in this example). A nickase complexes with the CRISPR guide RNA in close proximity to the protospacer adjacent motif (PAM) sequence to introduce a nick by cutting the “displaced strand” i.e., the strand opposite of the DNA recognition sequence. See, FIG. 1B.

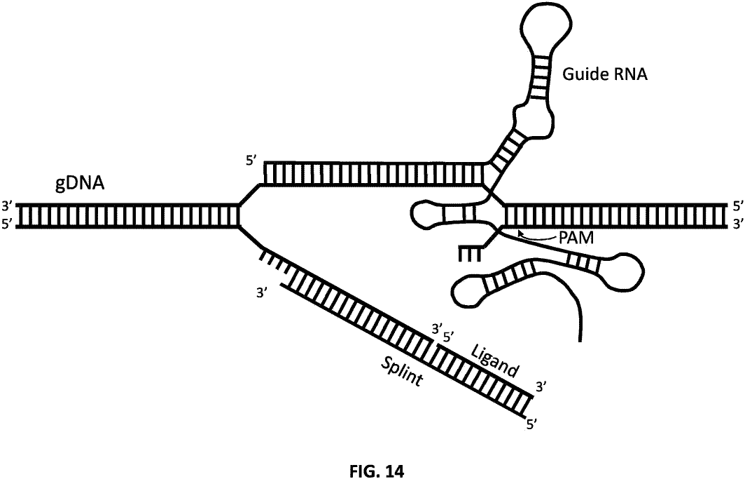

In one embodiment of the method as illustrated in FIG. 2, the CRISPR guide RNA targets a double-stranded genomic DNA (gDNA) near the intended editing site and a splint oligonucleotide hybridizes with the displaced strand. A ligand oligonucleotide hybridizes with the splint oligonucleotide. In another embodiment of the method as illustrated in FIG. 3, the CRISPR guide RNA hybridizes with the target sequence of the gDNA near the intended editing site, and a hairpin-shaped DNA structure including both the splint oligonucleotide and the ligand oligonucleotide binds to displaced strand of the double-stranded gDNA via the splint oligonucleotide sequence. FIG. 4 illustrates an embodiment of a hairpin-shaped DNA structure including both the splint oligonucleotide and the ligand oligonucleotide that also includes a photocleavable linker. The splint oligonucleotide ranges from about 10 bases to about 60 bases, sufficiently long to stabilize the duplex sufficiently for ligation to occur. The splint should be long enough to hybridize to both the displaced strand of the gDNA as well as the ligand. In embodiments using separate splint and ligand molecules (see e.g., FIG. 14), this length is usually 25-35 nucleotides, such as approximately 30 nucleotides. In embodiments using a hairpin-type molecule with the splint and the ligand (such as in FIG. 4, for example), the ‘splint’ part of the splint-ligand molecule can be shorter than the stand-alone splint, such as approximately 25 nucleotides. The length of the double-stranded region of the stem is determined by the distance to the variant site and the number of bases being altered. The intended base edits can be in the stem as indicated by colors in the Figures, or in the loop, depending on the distance between the intended editing site and the cleavage (nick) location.

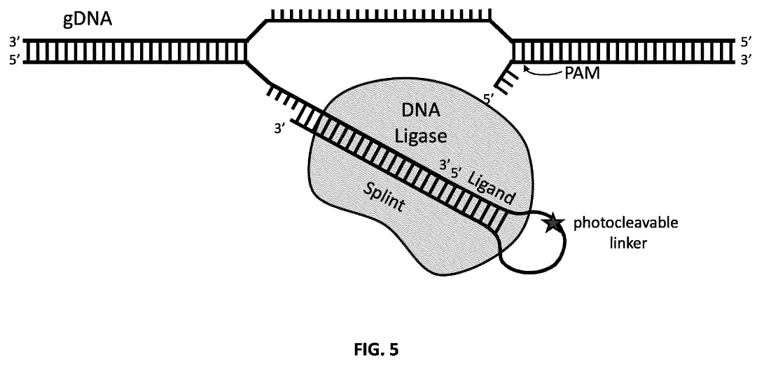

A DNA-ligase (illustrated in FIG. 5) ligates the ligand oligonucleotide to the genomic DNA, thus creating a heteroduplex with two flaps. The DNA-ligase can either be endogenous to the cell or, if necessary, exogenous and transfected into the cell.

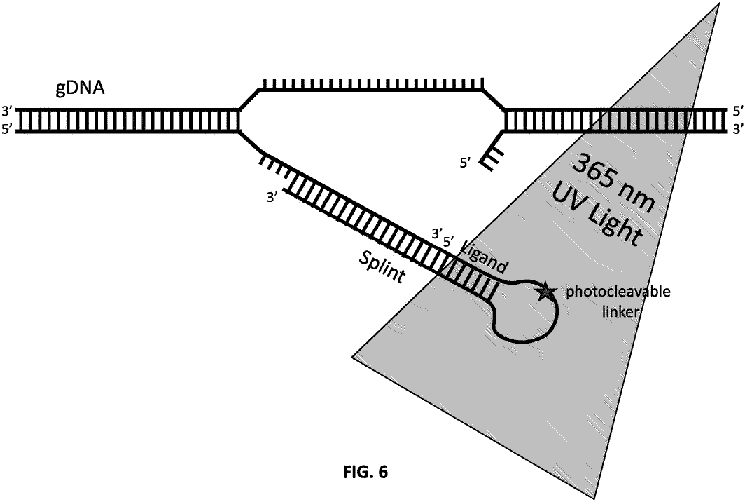

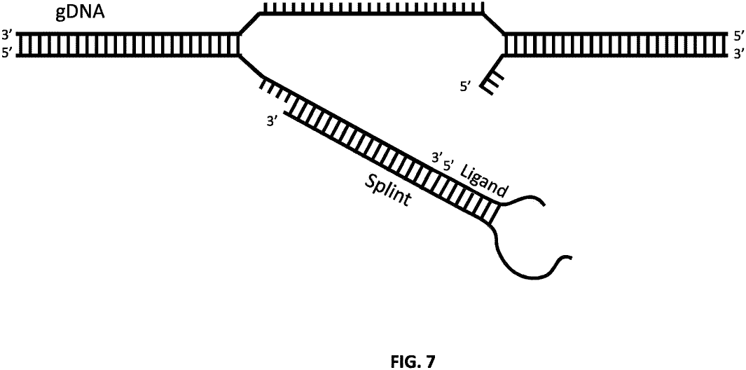

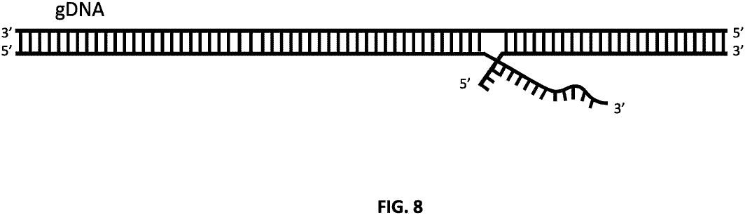

As illustrated in FIGS. 6 and 7, cleavage of the DNA hairpin by illuminating a photocleavable linker with UV-light (365 nm) destabilizes the hairpin structure. As illustrated in FIGS. 8 and 9, 5′ and 3′ flaps are cut such that the flaps are flush with the double-stranded nucleic acid. Flap cleavage occurs by a flap endonuclease.

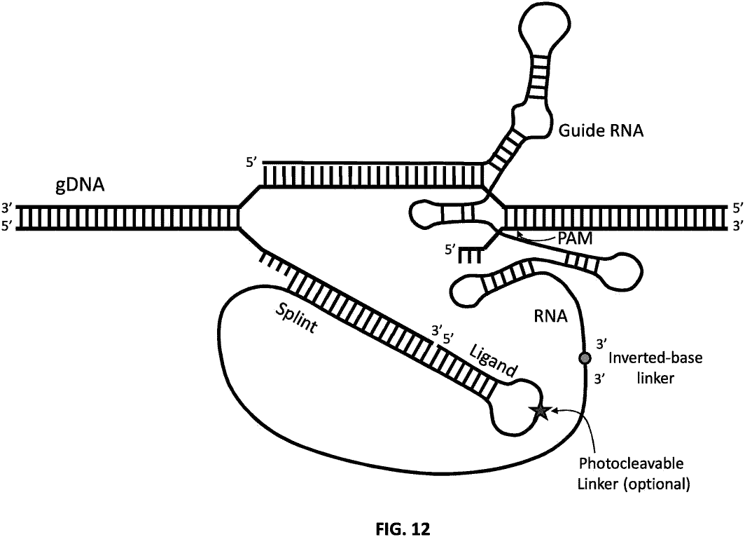

The embodiment shown in FIG. 12 concatenates the hairpin to the CRISPR guide RNA sequence using an inverted base near or at the concatenation site. An inverted base allows the 3′ end of a RNA guide sequence to be coupled to the 3′ end of a DNA hairpin sequence. Such inverted structures can be synthesized by those skilled in the art of polynucleotide synthesis. The inverted base can be at the junction of the RNA polynucleotide and the DNA polynucleotide, or within an RNA-RNA region or a DNA-DNA region. In the latter cases, the polynucleotide synthesis will need to transition the backbone from deoxyribonucleic to ribonucleic, or vice versa. Such methods are known to those skilled in the art of polynucleotide synthesis. The advantage of concatemerizing the splint oligonucleotide and/or ligand oligonucleotide to the CRISPR guide RNA is substantial since it ensures that there is high likelihood of there being a DNA ligand within close proximity to the cleaved end of the gDNA, without the need to transfect the cell with a high concentration of short DNA polynucleotides, which could be detrimental to the cell.

In some embodiments of the method, a ligand oligonucleotide can be concatemerized to the CRISPR guide RNA scaffold (as shown in FIG. 13), and the splint oligonucleotide is separated from the ligand oligonucleotide-CRISPR guide RNA concatemer. This method uses an inverted base and may optionally use a cleavable linker, such as a photocleavable linker, to liberate the short DNA strand and accelerate the digestion of the 3′ DNA flap after DNA-ligation.

As described herein, the CRISPR guide RNA, the splint oligonucleotide, the ligand oligonucleotide and/or the nickase can be transfected into cells independently or as an attached complex. The CRISPR guide RNA may be transfected into the cell using the same vector as for the nickase. In some embodiments, the splint oligonucleotide that spans the ligation junction is RNA rather than DNA.. In the case of an RNA splint, genomic DNA and ligand DNA form a heteroduplex of DNA and RNA, a special form of ligase sometimes called a splint-ligase can be used. This splint-ligase can ligate two DNA molecules in the form of a DNA/RNA hybrid duplex, where the RNA serves as the splint. As splint-ligase is not endogenous to mammalian cells, it will be transfected into the cytoplasm either as whole protein or (in a particularly suitable embodiment) attached with the Cas9 protein in the form of a fusion protein. Alternatively, it will be transfected as a vector into the cell and expressed. In either case, this fusion protein can be optimized using targeted mutagenesis to improve its ligation efficiency, specificity, or other important characteristics.

One key benefit of this architecture is that by attaching the splint-ligase to the Cas9, the ligation of the exogenous ligand-DNA to the genomic DNA should be accelerated and often precede the repair of the original nick introduced by the Cas9-nickase. Another advantage is that using an exogenous splint-ligase is expected to introduce fewer artifactual ligation events within the cell.

In another method illustrated in FIG. 15, the splint oligonucleotide can be an extension of the CRISPR guide RNA scaffold. This geometry has the useful advantage that no modification is required for the RNA, meaning that it can thus be transcribed from a vector DNA molecule. This embodiment utilizes a separate ligand oligonucleotide that is to be ligated to the target nucleic acid sequence.

FIGS. 12 and 16 represent equivalent geometries, except that FIG. 12 involves a hairpin loop structure, while FIG. 16 does not. In all of the embodiments where DNA is concatemerized to RNA, the transition point shown in the figure is merely illustrative. Both embodiments involve the conjugation of the DNA ligand with an RNA splint, as well as the use of a 3′-3′ inverted base to join the polynucleotides to the RNA guide sequence. The most effective transition point will be determined empirically. Optionally, a photocleavable linker can be used to shorten the DNA polynucleotide, making it more amenable to cleavage by the flap-endonuclease. FIG. 16 illustrates an inverted base as a linker between the splint oligonucleotide and the ligand oligonucleotide, and FIG. 12 illustrates an inverted base as a linker between the CRISPR guide RNA and the splint oligonucleotide. A splint-ligase is used to selectively ligate two DNA ends annealed to an RNA splint. Some bacteria are known to ligate DNA on an RNA splint, such as the PBCV-1 DNA ligase in the Chlorella virus (also known as Chlorella virus DNA Ligase). One such ligase that is commercially available is the SPLINTR®-Ligase from New England BIOLABS® Inc. (NEB).

In some embodiments, the CRISPR guide RNA, the splint oligonucleotide, the ligand oligonucleotide, and/or the Cas protein are incorporated into a vector that is transfected into the cell and expressed in the cell using methods known to those skilled in the art.

In some embodiments, the polynucleotides are modified to protect them from digestion by intracellular enzymes, or to accelerate their digestion or cleavage.

In some embodiments, the CRISPR guide RNA contains modifications to improve the specificity of the target site recognition over natural nucleotides. Further, all claims to sequences herein also applies to variants of sequences for improved efficiency or effectiveness.

The Cas nickase, the CRISPR guide RNA, the ligand oligonucleotide, the splint oligonucleotide, and combinations thereof, can be provided to a cell by transfection (e.g., chemical and lipid-mediated transfection), electroporation, physical deformation of a cell membrane, lipid nanoparticles (LNP), virus like particles (VLP), sonication, or any other method known in the art. Suitable electroporation-based delivery system systems include, for example, NUCLEOFACTOR/NUCLEOFECTION® (LONZA®), MaxCyte, Miltenyi CliniMACS, Neon electroporation, and BTX electroporation.

In other embodiments, each component can be separately produced in vitro and contacted (i.e., “complexed”) with each other in vitro to form a complex. The in vitro produced complexes can then be introduced (i.e., “delivered”) into a cell’s cytosol and/or nucleus.

In one aspect, the double-stranded nucleic acid for editing is the genome of a cell. Suitable cells include, for example, primary cells, stem cells (e.g., hematopoietic stem cells, embryonic stem cells), immune cells (e.g., T cells, natural killer cells, B cells, monocytes, macrophages, dendritic cells). Suitable cells include human cells, animal cells, other eukaryotic cells and bacterial cells.

Generally, the components used in the methods described herein form a complex to mediate sequence specific cleavage and ligation of a nucleic acid sequence to be incorporated into a double-stranded nucleic acid sequence.

In one aspect, the present disclosure is directed to a CRISPR guide RNA including a guide sequence that hybridizes with a target sequence; and at least one of a ligand oligonucleotide and a splint oligonucleotide. The guide sequence is complementary to a target sequence (also referred to herein as the “recognition sequence”).

The ligand oligonucleotide and CRISPR guide RNA can be coupled by at least one of an inverted-base linker and a cleavable linker.

The splint oligonucleotide and CRISPR guide RNA can be coupled by at least one of an inverted-base linker and a cleavable linker.

The guide sequence of the CRISPR guide RNA can range from about 17 nucleotides in length to about 30 nucleotides in length, including about 17 nucleotides to about 20 nucleotides, including about 18 nucleotides to about 25 nucleotides, and including about 21 nucleotides to about 24 nucleotides.

In one aspect, the present disclosure is directed to a CRISPR guide RNA including a guide sequence that hybridizes with a target nucleic acid sequence; at least one of an inverted base linker and a cleavable linker; and at least one of a ligand oligonucleotide and a splint oligonucleotide. In one embodiment, the present disclosure is directed to a CRISPR guide RNA including a guide sequence that hybridizes with a target nucleic acid sequence; and at least one of a ligand oligonucleotide and a splint oligonucleotide.

On the opposite strand of the target strand of the double-stranded nucleic acid is a protospacer adjacent motif (“PAM”). The PAM is a short DNA sequence that follows the DNA region to be cleaved by the nickase. The cut site is generally 3-4 nucleotide bases 5′ of the PAM site. The nickase to be used can be selected based on its PAM recognition sequence. Suitable nickases include Cas9 nickases.

The CRISPR guide RNA molecules can be generated synthetically or using recombinant expression. The CRISPR guide RNA can be synthetically generated by chemical synthesis. For recombinant expression, the CRISPR guide RNA is cloned into a plasmid vector that is introduced into a cell. The cell then uses its normal RNA polymerase to transcribe the genetic information from the plasmid vector to generate the CRISPR guide RNA. Another suitable method for generating the CRISPR guide RNA is by in vitro transcription. Generating the CRISPR guide RNA involves transcribing the CRISPR guide RNA from its corresponding DNA sequence outside a cell. The CRISPR guide RNA DNA sequence contains the RNA guide molecule sequence and an additional RNA polymerase promoter upstream of the CRISPR guide RNA sequence. The CRISPR guide RNA molecule is then transcribed using reagents and RNA polymerase.

In another aspect, the present disclosure is directed to a kit. The kit can include at least one of a CRISPR guide RNA, a splint oligonucleotide, a ligand oligonucleotide, a Cas nickase, and a Cas nickase-ligase fusion protein, and instructions for performing the method.

In one embodiment, the CRISPR guide RNA is coupled to at least one of the splint oligonucleotide, and ligand oligonucleotide. The CRISPR guide RNA, the splint oligonucleotide and/or the ligand oligonucleotide can be coupled via linkers such as, for example, one of an inverted base linker and a cleavable linker. In one embodiment, the splint oligonucleotide is coupled to the ligand oligonucleotide. The splint oligonucleotide and the ligand oligonucleotide can be coupled via linkers such as, for example, one of an inverted base linker and a cleavable linker.

The kits can also include a nickase. Suitable nickases are described herein.

The kits can also include an endonuclease. Suitable endonucleases include FEN1, for example.

The kits can include other components such as, for example, a ligase such as a DNA ligase and a splint ligase. The kits can also include nucleic acids and buffers.

The following is a short description of some embodiments of the present invention. The method and its variations described here involve using a CRISPR nickase to cleave genomic DNA near a target site. They rely on the activity of either endogenous or exogenous DNA ligase to incorporate an exogenous DNA ligand into the host genome, making a genomic alteration in a single strand. For the purpose of illustration, we will describe the use of a modified version of Cas9 that creates a nick (or single-strand cut) in the displaced DNA strand (that opposes the recognition strand) of the genomic DNA duplex. This method shares some features with Prime editing, such as the use of a multi-domain guide RNA and a Cas-nickase, but it does not utilize a reverse transcriptase to incorporate the intended edits into the genome.

Various embodiments of this invention are illustrated in the drawings. This method can be applied in living cells or in vitro, on biological or synthetic DNA. In addition to the Cas9 nickase, other nucleic acid polynucleotides are utilized, including three main components: the guide RNA (which comprises the CRISPR guide sequence and its scaffold sequence), a splint sequence, and a ligand sequence, described as follows:

The guide RNA sequence is any one of many natural or modified sequences that include a guide sequence that recognizes a genomic DNA sequence (“spacer”) near a PAM site and a scaffold RNA which is recognized by the CRISPR protein molecule.

The splint sequence is a template sequence which has a region complementary to the genomic DNA strand opposite to the CRISPR recognition sequence (spacer) and another region complementary to a sequence to be incorporated into the genome sequence by ligation. This polynucleotide can be either RNA or DNA, depending on the enzyme used to ligate the sequence to the genomic sequence.

The third polynucleotide is the ligand sequence. This comprises the sequence to be incorporated into the genome. Like the guide RNA and splint sequence, this polynucleotide may include natural bases or modified bases, such as methylated bases. This polynucleotide is typically between 10 nucleotide and 30 nucleotides in length, but as few as 1 (when concatenated to a guide) or as many as 100 nt. Other modifications may be used to increase its stability in the cell, provide one or more cleavage site(s), or enable its degradation after ligation. Methylated bases may be useful for applications where changing the methylation state of a genomic locus is useful.

In addition to these three polynucleotides, this invention depends on the activity of a DNA ligase. That DNA ligase can be a protein endogenous to the cell, exogenous to the cell and introduced as a vector sequence or added as a protein. Moreover, the DNA ligase can be introduced to the reaction as an individual component or concatenated to a Cas protein as a fusion protein. The modified Cas9 protein acts as a nickase, cutting a single strand of the genomic DNA duplex near the recognition sequence.

The invention of this disclosure may best be described by referring to the figures. FIG. 1B illustrates the effect of the nominal Cas9 guide structure and the cut induced in the genomic DNA by the Cas9 nickase, e.g., Cas9 H840A nickase, in close proximity to the PAM sequence on the strand opposite the DNA recognition sequence of the Cas9 guide. (This figure is illustrative of the architecture of Cas9 nickase. The binding between the RNA guide and the genomic DNA occurs only over the 20-bp targeting sequence.)

One embodiment of the method of this disclosure is illustrated in FIG. 2, which shows the guide RNA targeting the genomic DNA near the cut site, the ligand sequence, and the splint which hybridize to both the ligand and the spacer sequence 5′ of the single-stranded cut. The splint is shown as RNA but can be DNA or DNA-RNA chimera. The ligand is at least 8 bases in length, but can be 9, 10, 11, 12, 13, 14, 15, 16, 17, 18, 19, 20 bases or longer. In another embodiment (FIG. 3), the ligand and the splint are in the same molecule and has a hairpin-shaped DNA structure. The structure comprises a stem sequence, a loop structure optionally containing a cleavable linker, and a 3′ overhang complementary to the 3′ genomic flap that is on one side of the nick. Genome edits are designed into the ligand structure in the stem and/or loop regions. Following hybridization of the splint portion to the 3′ genomic DNA flap, the 5′ end of the ligand is ligated to the 3′ end of the genomic DNA through the action of a DNA ligase, and the loop is cleaved (FIG. 7). The DNA-ligase can either be endogenous to the cell, introduced as an individual component, or as a Cas9H840A-DNA Ligase fusion protein. The optimal range of overhang lengths between the hairpin stem and the genomic sequence is 5-20 bases, sufficiently long to stabilize the duplex sufficiently for ligation to occur. The length of the double-stranded region of the stem is determined by the distance to the variant site and the number of bases being altered.

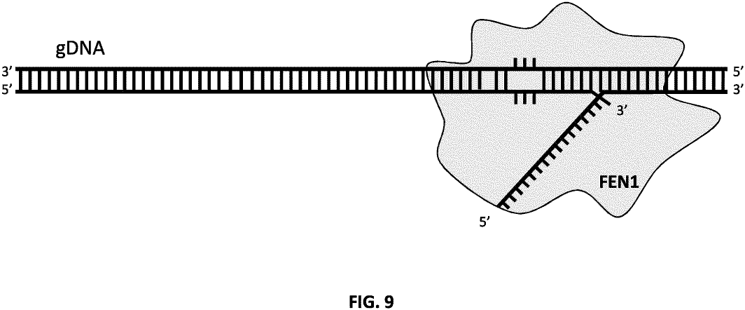

The remainder of the splint (the bottom oligonucleotide in FIG. 7) is removed. For example, if the splint is RNA, it may be automatically degraded in cells. Incorporation of the edited DNA sequence present on the flap resulting from the ligation of the ligand to the 3′ end of the genomic DNA will generally require the activity of a flap endonuclease (such as FEN1) as well as other general cellular DNA repair machinery, as described for PRIME editing (FIG. 9). For in vitro assays, FEN1 and DNA-ligase can be added to the reagent mix along with other reaction components.

FIG. 4 illustrates the incorporation of a photocleavable linker into the hairpin polynucleotide.

Cleavage of the DNA hairpin by means of illuminating a photo-cleavable linker with UV-light (such as 365 nm) will destabilize the hairpin structure accelerate the flap cleavage by flap endonuclease. The non-genomic bases can be in the stem as indicated by colors in the figure, or in the loop, depending on the distance between the targeted modification site and the cleavage location.

FIG. 12 illustrates an embodiment where a chimeric gRNA-ligand structure is used, with an inverted base near or at the chimera junction. An inverted base allows the 3′ end of the RNA guide sequence to be coupled to the 3′ end of the DNA hairpin ligand. Such inverted structures can be synthesized by those skilled in the art of polynucleotide synthesis. The inverted base can be at the junction of the RNA polynucleotide and the DNA polynucleotide, or within an RNA-RNA region or a DNA-DNA region. In the latter cases the polynucleotide synthesis will need to transition the backbone from deoxyribonucleic to ribonucleic, or vice versa. Such methods are known to those skilled in the art of polynucleotide synthesis. The advantage of concatemerizing the DNA to the RNA is substantial since it ensures that there is high likelihood of there being a DNA ligand within close proximity to the cleaved end of the gDNA, without the need to transfect the cell with a high concentration of short DNA polynucleotides, which could be detrimental to the cell.

In this geometry shown in FIG. 13, the DNA ligand is concatenated to the RNA guide scaffold, and the DNA splint polynucleotide is separated from the guide RNA-ligand structure. These molecules may be independently transfected into the cellular cytoplasm. This method requires an inverted base and may optionally utilize a cleavable linker, such as a photocleavable linker, to liberate the short DNA strand and accelerate the digestion of the 3′ DNA flap after DNA-ligation. In some embodiments the splint strand that spans the ligation junction is RNA rather than DNA. The RNA guide may be transfected into the cell using the same vector as for the Cas9-nickase. In the latter case, genomic DNA and ligand DNA form a heteroduplex of DNA and RNA, a special form of ligase sometimes called a splint-ligase is required and known to exist in nature [Lohman, 2019]. This splint-ligase will ligate two DNA molecules in the form of a DNA/RNA hybrid duplex, where the RNA serves as the splint. As splint-ligase is not endogenous to a mammalian cells, it can be transfected into the cytoplasm either as separate protein, as a Cas9-splint ligase fusion, or as a DNA expression vector. In any case, the splint ligase fusion protein can be optimized using targeted mutagenesis to improve its ligation efficiency, specificity, or other important characteristics. One key benefit of this architecture is that by attaching the splint-ligase to the Cas9, the ligation of the exogenous ligand-DNA to the genomic DNA should be accelerated and often precede the repair of the original nick introduced by the Cas9-nickase. Another key benefit is that using an exogenous splint-ligase is expected to introduce fewer artifactual ligation events within the cell. Another invention that also uses that gRNA to tether to a donor DNA polynucleotide is a patent application by Andrew Kennedy et al. (U.S. Pat. Application Pub. No. 2017/0058298, assignee Agilent Technologies).

FIG. 15 also includes an RNA splint sequence, but in this case the splint is an extension of the guide scaffold. This geometry has the useful advantage that no modification is required for the RNA, meaning that it can thus be transcribed from a vector DNA molecule. This embodiment utilizes a separate piece of DNA that is to be ligated to the genomic DNA, and this polynucleotide will need to be transfected into the cell. The DNA ligand in this case can be precomplexed with the gRNA prior to cell transfection. Once again, a splint ligase can be used for improved ligation specificity.

FIGS. 16 and 17 represent equivalent geometries, except that FIG. 17 involves a hairpin loop structure, while FIG. 16 does not. In all of the embodiments where DNA is concatenated to RNA, the transition point shown in the figure is merely illustrative. Both embodiments involve the conjugation of the DNA ligand with an RNA splint, as well as the use of a 3′-3′ inverted base to join the polynucleotides to the RNA guide sequence. The most effective transition point will be determined empirically. Optionally, a cleavable linker can be used to shorten the DNA polynucleotide making it more amenable to cleavage by the flap-endonuclease. FIG. 16 illustrates the inverted base as a linker between the RNA splint and the DNA ligand, and FIG. 17 illustrates the inverted base as a linker between the RNA guide and the RNA splint. Both embodiments can optionally utilize a splint-ligase to ligates two DNA ends with an RNA splint. Some bacteria are known to ligate DNA on an RNA splint, such as the PBCV-1 DNA ligase in the Chlorella virus. (See e.g., G.J. Lohman et al. NAR, 2014 Oct;42(18):11846; Y. Li and N. Peng. Front Microbiol. 2019; 10: 2471; S.B. Moon et al. Exp Mol Med. 2019 Nov; 51(11): 130; Smith, A. M. et al., Proc. Natl. Acad. Sci. USA 106, 5099-5104 (2009); Standage-Beier, K., Zhang, Q. & Wang, X., Acs Synth Biol 4, 1217-1225 (2015); and T. Yamano et al., Cell 165, 949-962 (2016)). One such ligase that is commercially available is the SPLINTR^{®}-Ligase from New England Biolabs.

To promote incorporation of the edited DNA sequence, an alternative embodiment (see FIG. 11) involves a second nicking event, on the same strand as the first nick, near the genomic position equivalent to the 3′ of the synthetic DNA molecule containing the edits. These two nicks are effected by the actions of Cas nickase enzymes targeting two nearby genomic positions. This method may not require an endogenous FEN1. Also, this nick may be repaired by the same enzyme that ligates the 5′-end of the ligand. As this method relies on the existence of a second PAM site the ligand must be made the appropriate length to reach the second genomic cleavage site. In this embodiment, the nickase used can be either one that cuts the target strand or that cuts the displaced strand.

In some embodiments, the guide structure, the template RNA, the Cas9 and the Cas9-ligase fusion protein vectors are incorporated into a vector that is transfected into the cell and expressed in the cell using methods known to those skilled in the art.

In some embodiments, the polynucleotides are modified to protect them from digestion by intracellular enzymes, or to accelerate their digestion or cleavage.

In some embodiments, the recognition polynucleotides are modified to improve the specificity of the target site recognition over natural nucleotides. Of course, all claims to sequences herein also applies to variants of sequences for improved efficiency or effectiveness.

The present invention can be practiced with any CRISPR system for which nicking variants are presently or eventually available, including but not limited to those based on cas9 and cas12a. A modification of the Cas12a protein has been demonstrated nickase activity with cleavage of the non-targeted strand, indicating that this enzyme can be used in this assay, although as the scaffold is on the 5′-end of the guide the geometry is different than those shown in the drawings for Cas9.

CRISPR systems are known in the art. For example, the references cited herein are incorporated by references in their entirety, particularly with respect to the description of structure and function of CRISPR proteins, CRISPR nickases, and guide RNAs.

### EXEMPLARY EMBODIMENTS

Exemplary Embodiment 1. A method of editing a double-stranded nucleic acid comprising a target strand and a displaced strand that is complementary to the target strand, the method comprising:
providing to the double-stranded nucleic acid:
   a Cas nickase;
   a CRISPR guide RNA comprising a guide sequence that hybridizes with the target strand of the double-stranded nucleic acid;
   a ligand oligonucleotide comprising a nucleic acid sequence to be incorporated into the double-stranded nucleic acid;
   a splint oligonucleotide comprising a first nucleic acid sequence that hybridizes with the ligand oligonucleotide and a second nucleic acid sequence that hybridizes with the displaced strand of the double-stranded nucleic acid;
   wherein the CRISPR guide RNA and Cas nickase form a complex with the double-stranded nucleic acid and make a single-strand cut in the displaced strand to form a nick in the displaced strand, and the splint oligonucleotide hybridizes to the displaced strand adjacent to the nick, and the ligand oligonucleotide hybridizes to the splint oligonucleotide; and
ligating one end of the ligand oligonucleotide to a nucleotide adjacent to the nick in the displaced strand to thereby result in incorporation of the ligand oligonucleotide into the double-stranded nucleic acid.

Exemplary Embodiment 2. The method of Exemplary Embodiment 1, wherein the splint oligonucleotide is coupled to the CRISPR guide RNA.

Exemplary Embodiment 3. The method of Exemplary Embodiment 1 or 2, wherein the ligand oligonucleotide is coupled to the CRISPR guide RNA.

Exemplary Embodiment 4. The method of Exemplary Embodiment 1, wherein the ligand oligonucleotide and the splint oligonucleotide are coupled to the CRISPR guide RNA.

Exemplary Embodiment 5. The method of Exemplary Embodiment 4, wherein the CRISPR guide RNA further comprises a hairpin.

Exemplary Embodiment 6. The method of any one of the Exemplary Embodiment s, wherein at least two of the ligand oligonucleotide, the splint oligonucleotide, and the CRISPR guide RNA are coupled by a linker.

Exemplary Embodiment 7. The method of Exemplary Embodiment 6, wherein the linker is any one of an inverted base linker, a cleavable linker, and combinations thereof.

Exemplary Embodiment 8. The method of Exemplary Embodiment 1, wherein the ligand oligonucleotide and the splint oligonucleotide are coupled.

Exemplary Embodiment 9. The method of Exemplary Embodiment 8, wherein the ligand oligonucleotide and the splint oligonucleotide are coupled, and further comprise a hairpin.

Exemplary Embodiment 10. The method of any one of Exemplary Embodiments 8 and 9, wherein the ligand oligonucleotide and the splint oligonucleotide are coupled by a linker.

Exemplary Embodiment 11. The method of Exemplary Embodiment 10, wherein the linker is any one of an inverted base linker, a cleavable linker, and combinations thereof.

Exemplary Embodiment 12. The method of any one of the Exemplary Embodiments, further comprising illuminating with ultraviolet light.

Exemplary Embodiment 13. The method of any one of the Exemplary Embodiments, further comprising providing a DNA ligase.

Exemplary Embodiment 14. The method of Exemplary Embodiment 13, wherein the DNA ligase is selected from T4 ligase, T7 ligase, T3 ligase, and PBCV-1 DNA Ligase.

Exemplary Embodiment 15. The method of any one of the preceding paragraphs, further comprising providing a flap endonuclease.

Exemplary Embodiment 16. The method of any one of the Exemplary Embodiments, wherein the double-stranded nucleic acid is in a cell in a sample.

Exemplary Embodiment 17. The method of any one the Exemplary Embodiments, wherein the Cas nickase comprises a fusion protein.

Exemplary Embodiment 18. The method of Exemplary Embodiment 17, wherein the fusion protein comprises a ligase.

Exemplary Embodiment 19. The method of any one of the Exemplary Embodiments, wherein the splint oligonucleotide is a DNA, an RNA, and combinations thereof.

Exemplary Embodiment 20. The method of any one of the Exemplary Embodiments, further comprising: providing a second CRISPR guide RNA that comprises from 5′ to 3′: a guide sequence that hybridizes with a second nucleic acid sequence in the target strand of the double-stranded nucleic acid that is different from the nucleic acid sequence to which the first CRISPR guide RNA hybridizes.

Exemplary Embodiment 21. A CRISPR guide RNA comprising: a guide sequence that hybridizes with a target nucleic acid sequence of a double-stranded nucleic acid sequence; and at least one of a ligand oligonucleotide and a splint oligonucleotide.

Exemplary Embodiment 22. The CRISPR guide RNA of Exemplary Embodiment 21, wherein the ligand oligonucleotide and CRISPR guide RNA are coupled by at least one of an inverted-base linker and a cleavable linker.

Exemplary Embodiment 23. The CRISPR guide RNA of Exemplary Embodiment 21, wherein the splint oligonucleotide and CRISPR guide RNA are coupled by at least one of an inverted-base linker and a cleavable linker.

Exemplary Embodiment 24. The method or CRISPR guide RNA of any one of the preceding Exemplary Embodiments, wherein the guide sequence ranges from about 17 nucleotides to about 25 nucleotides.

Exemplary Embodiment 25. The CRISPR guide RNA of any one of Exemplary Embodiments 21-24, wherein the splint oligonucleotide sequence is a DNA, a RNA, and combinations thereof.

Exemplary Embodiment 26. The CRISPR guide RNA of any one of Exemplary Embodiments 21-25, wherein the ligand oligonucleotide sequence is a DNA.

Exemplary Embodiment 27. The method or CRISPR guide RNA of any one of the preceding Exemplary Embodiments, wherein the splint oligonucleotide is an RNA and the ligand oligonucleotide is a DNA.

Exemplary Embodiment 28. The method or CRISPR guide RNA of any one of the preceding Exemplary Embodiments, wherein the splint oligonucleotide is a DNA and the ligand oligonucleotide is a DNA.

Exemplary Embodiment 29. The method or CRISPR guide RNA of any one of the preceding Exemplary Embodiments, wherein the CRISPR guide RNA and splint RNA are coupled by an inverted-base linker.

Exemplary Embodiment 30. The CRISPR guide RNA of any one of Exemplary Embodiments 21-29, wherein the ligand oligonucleotide sequence and splint oligonucleotide sequence are coupled by an inverted-base linker, a cleavable linker, and combinations thereof.

Exemplary Embodiment 31. The method or CRISPR guide RNA of any one of the preceding Exemplary Embodiments, wherein the CRISPR guide RNA is attached to a ligase.

Exemplary Embodiment 32. The method or CRISPR guide RNA of any one of the preceding Exemplary Embodiments, wherein the CRISPR guide RNA is labeled with biotin to recruit a ligase that is capable of binding strepavidin.

Exemplary Embodiment 33. A kit for ligation-based gene editing comprising: a CRISPR guide RNA of any one of the preceding Exemplary Embodiments; and at least one of a ligand oligonucleotide and a splint oligonucleotide; and instructions for performing ligation-based gene editing.

Exemplary Embodiment 34. The method, CRISPR guide RNA or kit of any one of the preceding Exemplary Embodiments in which the ligand oligonucleotide and the splint oligonucleotide are not coupled, wherein the ligand oligonucleotide and the splint oligonucleotide are pre-hybridized.

Exemplary Embodiment 35. A method of editing a target nucleic acid in a sample, comprising:
providing the following to the sample:
   a CRISPR nickase;
   a CRISPR guide RNA comprising a guide sequence that recognizes a target sequence in the target nucleic acid, to result in a nick in the opposite strand of the target sequence by the CRISPR nickase,
   a ligand oligonucleotide comprising a ligand sequence to be incorporated into the target nucleic acid,
   a splint oligonucleotide comprising a first sequence complementary to the ligand sequence, and a second sequence complementary to the opposite strand of the target sequence adjacent the nick; and
causing the nick to be ligated and the ligand sequence incorporated into the target nucleic acid.

Exemplary Embodiment 36. The method of Exemplary Embodiment 35, wherein the ligand oligonucleotide and the splint oligonucleotide are in the same molecule.

Exemplary Embodiment 37. The method of Exemplary Embodiment 35, wherein the molecule is the guide RNA.

Exemplary Embodiment 38. The method of Exemplary Embodiments 35 and 36, wherein the ligand oligonucleotide and the splint oligonucleotide are at an end of the guide RNA distal from the guide sequence.

Exemplary Embodiment 39. The method of Exemplary Embodiments 35, 36, 37, or 38, wherein the molecule comprises a cleavable linker between the ligand oligonucleotide and the splint oligonucleotide.

Exemplary Embodiment 40. The method of Exemplary Embodiment 39, wherein the cleavable linker is a photocleavable linker.

Exemplary Embodiment 41. The method of Exemplary Embodiment 35, wherein the ligand oligonucleotide, but not the splint oligonucleotide, is part of the guide RNA.

Exemplary Embodiment 42. The method of Exemplary Embodiment 35, wherein the splint oligonucleotide, but not the ligand oligonucleotide, is part of the guide RNA.

Exemplary Embodiment 43. The method of any one of the preceding Exemplary Embodiments, further comprising providing a ligase to the sample.

Exemplary Embodiment 44. The method of any one of the preceding Exemplary Embodiments, further comprising providing a flap nuclease to the sample.

Exemplary Embodiment 45. The method of any one of the preceding Exemplary Embodiments, wherein the sample is a cell.

Exemplary Embodiment 46. The method of Exemplary Embodiment 45, wherein the cell is a primary cell.

Exemplary Embodiment 47. The method of any one of the preceding Exemplary Embodiments, wherein the CRISPR nickase is provided as a fusion protein that also comprises a ligase.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments and the claims. Reference should therefore be made to the embodiments and claims for interpreting the scope of the present disclosure.

### EXAMPLES

### Example 1

In this example, configured as in FIG. 2, the method of gene editing was performed in vitro:

Step 1: 10 picomoles each of the splint and ligand nucleic acids were combined in a solution of 100 mM KCl and 50 mM Tris-Cl pH 7.5 and annealed by incubating at 37° C. for 20 minutes.

Step 2: a ribonucleoprotein (RNP) was prepared by combining 2.5 picomoles of sgRNA with 250 nanograms of Cas9H840A protein in a total volume of 5 microliters. This was incubated for 5 minutes at 25° C.

Step 3: The remaining reaction components were added to the RNP prepared above to a final volume of 25 microliters:
5 microliters 5× reaction buffer (250 mM Tris-Cl pH 7.5, 4 mM MgCl₂, 50 mM NaCl, 700 mM KCl);
62.5 femtomoles of double-stranded target DNA;
1 pmol annealed splint+ligand (see Step 1); and
3000 units of T3 DNA Ligase (New England Biolabs).

The above reaction mixture was incubated at 37° C. for 60 minutes.

Step 4: The reaction product was purified using AMPure XP beads (Beckman Coulter) according to the manufacturer’s recommendations.

Step 5: Editing can be detected and quantified in at least two ways. The resulting reaction product can be run on a denaturing Urea-PAGE gelor the edited target sequence can be amplified with PCR using a primer binding to a sequence present on the ligand.

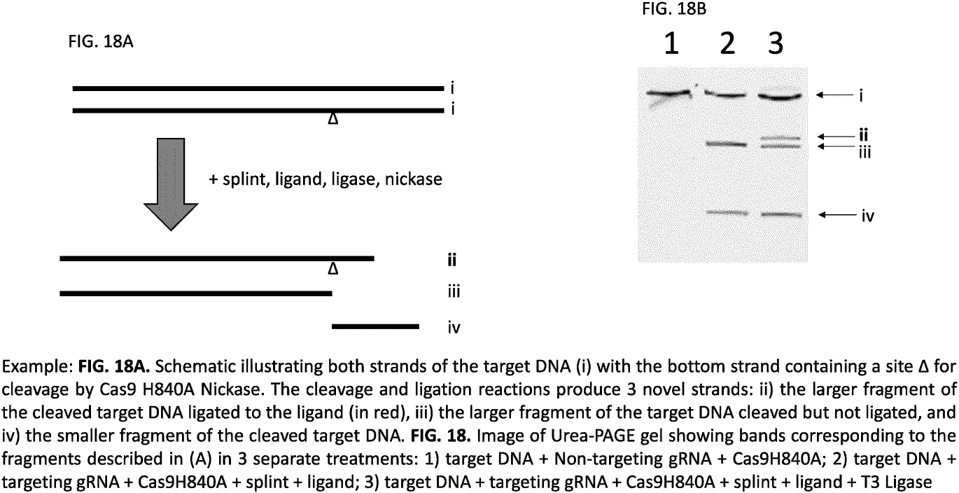

FIG. 18 shows the results of detection by a gel, which indicate that the ligand was incorporated into the target as expected. FIG. 18A illustrates the expected results, wherein fragments iii and iv are the products of a nick at the expected place, and fragment ii is the result of incorporation of the ligand. FIG. 18B demonstrates that the actual results are as expected.

### Example 2

In this Example, the method of gene editing is performed in K562 cells.

Step 1: 500 picomoles each of the splint and ligand nucleic acids are combined in a solution of 100 mM KCl and 50 mM Tris-Cl pH 7.5 and annealed by incubating at 37° C. for 20 minutes.

Step 2: a ribonucleoprotein (RNP) is prepared by combining 125 picomoles of sgRNA with 50 picomoles of Cas9H840A-T3 DNA Ligase fusion protein in a total volume of 10 microliters. This is incubated for 5 minutes at 25° C.

Step 3: The remaining components are added to the RNP prepared above to a final volume of 25 microliters:
200,000 K562 cells
3000 units of T3 DNA Ligase (New England Biolabs)
100 picomoles of annealed splint+ligand (see Step 1)

Step 4: The cell suspension is then transfected by using a 4D nucleofection system (Lonza).

Step 5: The transfected cells are grown in 1 milliliter RPMI medium supplemented with 10% fetal bovine serum for 48 hours.

Step 6: Editing is quantified using amplicon-based next generation sequencing over the targeted region. Genomic DNA is isolated from cells, targeted region is amplified by PCR, and with appropriate sequencing adaptors, the amplified product is sequenced.

The sequencing results are expected to show that the ligand sequences are incorporated into the target site. The experimental protocol and conditions can deviate from those described in this Example 2 based on ordinary skills in the art to obtain the expected results.

1. A method of editing a double-stranded nucleic acid comprising a target strand and a displaced strand that is complementary to the target strand, the method comprising:
providing to the double-stranded nucleic acid:
a Cas nickase;
a CRISPR guide RNA comprising a guide sequence that hybridizes with the target strand of the double-stranded nucleic acid;
a ligand oligonucleotide comprising a nucleic acid sequence to be incorporated into the double-stranded nucleic acid;
a splint oligonucleotide comprising a first nucleic acid sequence that hybridizes with the ligand oligonucleotide and a second nucleic acid sequence that hybridizes with a nucleic acid sequence in the displaced strand of the double-stranded nucleic acid;
wherein the CRISPR guide RNA and Cas nickase form a complex with the double-stranded nucleic acid and make a single-strand cut in the displaced strand to form a nick in the displaced strand, and the splint oligonucleotide hybridizes to the displaced strand adjacent to the nick, and the ligand oligonucleotide hybridizes to the splint oligonucleotide; and
ligating one end of the ligand oligonucleotide to a nucleotide adjacent to the nick in the displaced strand to thereby result in incorporation of the ligand oligonucleotide into the double-stranded nucleic acid.

2. The method of claim 1, wherein the splint oligonucleotide is coupled to the CRISPR guide RNA.

3. The method of claim 1, wherein the ligand oligonucleotide is coupled to the CRISPR guide RNA.

4. The method of claim 1, wherein at least two of the ligand oligonucleotide, the splint oligonucleotide, and the CRISPR guide RNA are coupled by a linker.

5. The method of claim 4, wherein the linker is any one of an inverted base linker, a cleavable linker, and combinations thereof.

6. The method of claim 1, wherein the ligand oligonucleotide and the splint oligonucleotide are coupled.

7. The method of claim 1, further comprising providing a DNA ligase.

8. The method of claim 7, wherein the DNA ligase is selected from T4 ligase, T7 ligase, T3 ligase, and PBCV-1 DNA Ligase.

9. The method of claim 7, wherein the DNA ligase and the Cas nickase are provided in a fusion protein.

10. The method of claim 1, further comprising providing a flap endonuclease.

11. The method of claim 1, wherein the double-stranded nucleic acid is in a cell in a sample.

12. The method of claim 1, wherein the splint oligonucleotide is a DNA, an RNA, or combinations thereof.

13. The method of claim 1, further comprising: providing a second CRISPR guide RNA that comprises from 5′ to 3′: a guide sequence that hybridizes with a second nucleic acid sequence in the target strand of the double-stranded nucleic acid that is different from the nucleic acid sequence to which the first CRISPR guide RNA hybridizes.

14. A CRISPR guide RNA comprising:
a guide sequence that hybridizes with a target nucleic acid sequence of a double-stranded nucleic acid sequence; and
at least one of a ligand oligonucleotide and a splint oligonucleotide.
